(19) United States
(12) Patent Application Publication
(10) Pub. No.: US 2024/0122758 A1
(43) Pub. Date: Apr. 18, 2024

(54) METHOD FOR PROVIDING CONTROL DATA FOR PRESBYOPIA REVERSION FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventors: Samuel ARBA MOSQUERA, Aschaffenburg (DE); Tobias EWERING, Kleinwallstadt (DE)

(21) Appl. No.: 18/474,707

(22) Filed: Sep. 26, 2023

(30) Foreign Application Priority Data
Oct. 14, 2022 (DE) 10 2022 126 855.1

(51) Int. Cl. A61F 9/008

(52) U.S. Cl. CPC A61F 9/00808; A61F 9/00827; A61F 2009/00878; A61F 2009/00895

(57) ABSTRACT

The invention relates to a method for providing control data for presbyopia reversion for an ophthalmological laser (12) of a treatment apparatus (10). As steps, the method includes determining (S10) presbyopia correction data for presbyopia correction of a cornea (16), wherein an originally uniform visual acuity of the cornea (16) is changed into multifocal areas by application of the presbyopia correction data, wherein the presbyopia correction data is stored in a database (24); retrieving (S12) the stored presbyopia correction data of the cornea (16) from the database (24), if the multifocal areas of the cornea (16) are to be adapted or cancelled at a subsequent point of time; determining (S14) control data for adapting or cancelling the multifocal areas depending on the retrieved presbyopia correction data; and providing (S16) the control data for controlling the ophthalmological laser (12) of the treatment apparatus (10).

### FIELD

The invention relates to a method for providing control data for presbyopia reversion for an ophthalmological laser of a treatment apparatus. In addition, the invention relates to a method for controlling a treatment apparatus, to a treatment apparatus with at least one ophthalmological laser and at least one control device for performing one of the methods, to a computer program and to a computer-readable medium.

### BACKGROUND

Treatment apparatuses and methods for controlling ophthalmological lasers for the correction of an optical visual disorder and/or pathologically or unnaturally altered areas of the cornea are known in the prior art. Therein, a pulsed laser and a beam focusing device can for example be formed such that laser pulses effect a photodisruption and/or photoablation in a focus situated within the organic tissue to remove a tissue, in particular a tissue lenticule, from the cornea. For removing the tissue, it is required to provide suitable control data, by which it is indicated, in which positions of the cornea laser pulses have to be applied to achieve the desired treatment success.

Therein, a very challenging correction of the cornea is the presbyopia correction (correction of presbyopia), which occurs due to an age-related loss of the near adaptation capability of the eye. For correcting the presbyopia, glasses can be manufactured, in particular varifocal glasses, which comprise multiple areas of different refractive power. A further possibility are multifocal laser treatments of the cornea, wherein one or more areas of different refractive power are generated in the cornea in these methods in similar manner as in varifocal glasses.

One of the greatest concerns in this induced multifocal presbyopia correction is the reversibility of the procedure to again achieve a monofocal cornea if a patient has problems with the induced multifocal cornea. Such multifocal optical systems cannot be completely corrected by conventional glasses or contact lenses. In case of the multifocal ablation, it is not clear if multifocal corneas can be resolved by either Placido-based topography or Hartmann-Shack aberrometry and can be reconverted into normal corneas by a wavefront-driven approach.

It is the object of the invention to achieve an improved reversion of a presbyopia correction of a cornea.

### SUMMARY

This object is solved by the independent claims. Advantageous configurations with convenient developments of the invention are specified in the respective dependent claims, wherein advantageous configurations of the method are to be regarded as advantageous configurations of the treatment apparatus, of the control device, of the computer program and of the computer-readable medium and vice versa.

The invention is based on the idea that the presbyopia reversion for compensating for the multifocality is completely planned on the treatment data, which has been used for the original presbyopia correction.

A first aspect of the invention relates to a method for providing control data for the presbyopia reversion for an ophthalmological laser of a treatment apparatus. As steps, the method includes determining presbyopia correction data for presbyopia correction of a cornea, wherein an originally uniform visual acuity of the cornea is changed into multifocal areas by application of the presbyopia correction data, wherein the presbyopia correction data is stored in a database, retrieving the stored presbyopia correction data of the cornea from the database if the multifocal areas of the cornea are to be adapted or cancelled at a subsequent point of time, determining control data for adapting or cancelling the multifocal areas depending on the retrieved presbyopia correction data, and providing the control data for controlling the ophthalmological laser of the treatment apparatus.

In other words, presbyopia correction data can be ascertained, which defines multifocal areas in the cornea, to perform a presbyopia correction in a patient. This originally determined presbyopia correction data can be stored in a database, wherein the database is preferably a non-volatile storage, preferably in a computer cloud. Preferably, the database can be protected from data loss by means of usual mechanisms such that the originally determined presbyopia correction data can be retrieved and/or restored for a preset period of time.

For a patient, who has been treated by means of the presbyopia correction data and whose cornea comprises the multifocal areas, it can occur that the multifocal areas are annoying and/or erroneous, wherein the patient wishes to cancel them and to return to a monofocal cornea. In this case, the stored presbyopia correction data can be loaded from the database upon treatment planning for reversing the presbyopia correction. Therein, the determination of the control data for the presbyopia reversion can be performed exclusively on these retrieved presbyopia correction data of the original treatment without performing an additional measurement of the cornea by means of measurement apparatuses. Herein, modifications on the cornea can be performed based on the original presbyopia correction data such that the original treatment is inverted. This means that a multifocality of the cornea is again removed or is reset to a state before the presbyopia correction. For example, an original curvature progression of the cornea can be restored in that areas are for example ascertained in the presbyopia correction data, which have not been treated or removed in the original treatment. These areas can then be marked as the tissue to be treated in the control data for compensating for the multifocality to undo the presbyopia correction and in particular to restore the curvature progression as before the presbyopia correction. Alternatively, only the multifocality can also be eliminated and a curvature progression of a refraction correction can be maintained.

Therein, the presbyopia correction does not have to be completely reversed since only a too large order of magnitude of a refractive power change in the cornea is often perceived as uncomfortable by the patient, wherein it can then be mitigated with the aid of the presbyopia correction data. The thus ascertained control data for adapting or cancelling the multifocal areas of the presbyopia correction can then be provided to the ophthalmological laser of the treatment apparatus for presbyopia reversion.

For example, the method can be performed by a control device, which ascertains the presbyopia correction data in the original treatment and can store it on a database. For the reversion of the presbyopia correction, the control device can retrieve the data from the database, wherein this data can then be processed by means of suitable adaptations for cancelling or adapting the presbyopia correction.

By the invention, the advantage arises that an improved reversion of the presbyopia correction can be achieved. In particular, measurement apparatuses for measuring the cornea, such as for example a video keratoscope, cannot effectively determine different multifocal areas and the primary and secondary spherical aberrations induced thereby, which can result in an erroneous reversion of the presbyopia.

The invention also includes forms of configuration, by which additional advantages arise.

A form of configuration provides that the multifocal areas preset from the presbyopia correction data are adapted by at least partially compensating for these multifocal areas. In other words, the radius of curvature can be partially changed to the original curvature in these areas. Herein, it can be provided that not the entire cornea is again reversed to the original uniform visual acuity, but that only a part of the multifocal areas is undone, in particular by a preset refractive power. This means that not the complete original curvature has to be restored in these areas, but the multifocality can only be mitigated, for example from 2 diopters to 1 diopter. Thus, an original effect of the presbyopia correction can be mitigated, which is already tolerable for many patients. Thus, there is preferably also the possibility of adapting further multifocal areas up to the complete compensation for the presbyopia correction in further subsequent treatments.

A further form of configuration provides that the multifocal areas preset from the presbyopia correction data are adapted by complete compensation for the multifocal areas to the original uniform visual acuity. This means that the original curvature of the entire cornea is restored in that the original presbyopia correction is inverted. Thus, the presbyopia correction can be completely reversed by suitable removal of areas, which can be ascertained from the presbyopia correction data.

Preferably, it is provided that the presbyopia correction data includes information about an optical zone and a pupil-to-vertex offset. In other words, the original multifocal planning, in particular the optical zone together with the pupil-to-vertex offset, can be present for a successful presbyopia reversion to provide a successful reversion of the presbyopia correction.

Particularly preferably, it is provided that corneal tissue outside of the optical zone is determined in the control data for compensating for multifocal areas. In particular, a compensation can be made for multifocal areas, which have been provided by change of the optical zone, in that non-treated areas, which are located outside of the optical zone, are removed. Thus, a radius of curvature of the cornea, which was present before the original presbyopia correction, can be restored.

In a further advantageous form of configuration, it is provided that the pupil-to-vertex offset is changed in the control data for compensating for a coma aberration. Upon a presbyopia correction, an undesired coma aberration can in particular be induced, which becomes noticeable for a patient only after the treatment. In order that this coma aberration is not also present after the reversion of the presbyopia correction, a centering can preferably be adapted in that the pupil-to-vertex offset is compensated for. Thus, an improved treatment result can be achieved.

A further aspect of the invention relates to a method for controlling a treatment apparatus. Therein, the method includes the method steps of at least one embodiment of the method as it was previously described. Furthermore, the method for controlling the treatment apparatus also includes the step of transmitting the provided control data to at least one eye surgical or ophthalmological laser of the treatment apparatus. Then, the treatment apparatus and/or the laser can be controlled for generating a pattern in the cornea by means of the control data.

The respective method can include at least one additional step, which is executed if and only if an application case or an application situation occurs, which has not been explicitly described here. The step can for example include the output of an error message and/or the output of a request for inputting a user feedback. Additionally or alternatively, it can be provided that a default setting and/or a predetermined initial state is adjusted.

A further aspect of the invention relates to a control device, which is formed to perform the steps of at least one embodiment of one or both of the previously described methods. Thereto, the control device can comprise a computing unit for electronic data processing such as for example a processor. The computing unit can include at least one microcontroller and/or at least one microprocessor. The computing unit can be designed as an integrated circuit and/or microchip. Furthermore, the control device can include an (electronic) data storage or a storage unit. A program code can be stored on the data storage, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. For example, the control device can be included by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser can be formed to at least partially separate one or more predefined cut surfaces in the cornea, in particular a corneal volume with predefined interfaces of a human or animal eye, by means of optical breakthrough, in particular at least partially separate them by means of photodisruption and/or to ablate corneal layers by means of ablation and/or to effect a laser-induced refractive index change in the cornea and/or the eye lens.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

In further advantageous configurations of the treatment apparatus according to the invention, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. For example, the storage medium is formed as a data storage, in particular at least partially as a volatile or non-volatile data storage. A non-volatile data storage can be a flash memory and/or an SSD (solid state drive) and/or a hard disk. A volatile data storage can be a RAM (random access memory). The commands can for example be present as a source code of a programming language and/or as an assembler and/or as a binary code.

Further features and advantages of one of the described aspects of the invention can result from the developments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described based on the figure(s) in the form of advantageous execution examples. The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown in the figures or explained, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims.

FIG. 1 is a schematic representation of a treatment apparatus according to an exemplary embodiment.

FIG. 2 is a schematic method diagram for providing control data for presbyopia reversion according to an exemplary embodiment.

In the figures, identical or functionally identical elements are provided with the same reference characters.

### DETAILED DESCRIPTION

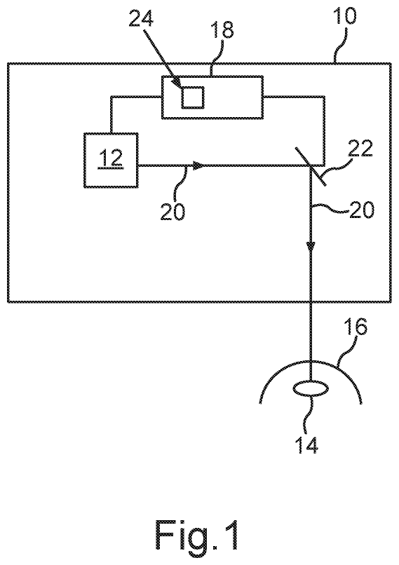

FIG. 1 shows a schematic representation of a treatment apparatus 10 with an eye surgical laser 12 for the treatment of a cornea 16 by means of photodisruption and/or ablation. For treatment of the cornea 16, treatment positions, in particular within an optical zone 14, on which a cavitation bubble path for separating tissue from the cornea 16 can be generated, are preset in the control data. One recognizes that a control device 18 for the laser 12 can be formed besides the laser 12 such that it can emit pulsed laser pulses for example in a predefined pattern. Alternatively, the control device 18 can be a control device 18 external with respect to the treatment apparatus 10.

Furthermore, the FIG. 1 shows that the laser beam 20 generated by the laser 12 is deflected towards the cornea 16 by means of a beam device 22, namely a beam deflection device such as for example a rotation scanner. The beam deflection device 22 is also controlled by the control device 20 to treat the cornea 16.

Preferably, the illustrated laser 12 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. The control device 18 additionally comprises a storage device 24 for storing at least one control dataset, wherein the control dataset or datasets include(s) control data for positioning and/or for focusing individual laser pulses in the cornea 16. Here, the storage device is illustrated as a part of the control device 18, however, the storage device can also be provided as an external storage, in particular in the form of a computer cloud. The position data and/or focusing data of the individual laser pulses, in particular for a presbyopia correction, can be generated based on predetermined measurements, for example from a previously measured topography and/or pachymetry and/or the morphology of the cornea or the optical visual disorder correction to be generated.

For determining the visual disorder data, which can for example indicate a value in diopters, suitable examination data for describing the visual disorder can be received by the control device 18 from a data server or the examination data can be directly input into the control device 18.

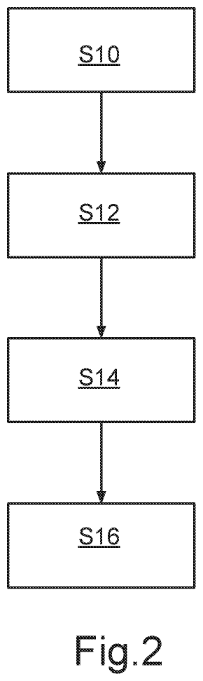

Preferably, the treatment apparatus 10 can be formed to provide presbyopia correction data for a presbyopia correction of the cornea 16 and also to perform a reversion of this induced presbyopia correction. Thereto, the control device 18 can for example perform a method, which is schematically illustrated in FIG. 2.

In a step S10, presbyopia correction data for presbyopia correction of the cornea 16 can be ascertained, by which an original uniform visual acuity of the cornea 16 is changed into multifocal areas, in particular within the optical zone 14. At least the information about the optical zone 14 and information about a pupil-to-vertex offset can be provided in the presbyopia correction data, which is applied for the presbyopia correction. This presbyopia correction data can be stored in the storage device 24, in particular a database of the storage device 24. After the presbyopia correction has been performed by means of the presbyopia correction data, in that the laser 12 has incorporated multifocal areas into the optical zone 14 of the cornea 16 according to the presbyopia correction data, it can be provided in a subsequent treatment that the multifocal areas are to be undone. For example, it can occur if they are unsuitable or annoying for a patient.

Therefore, a subsequent treatment can be provided in a step S12 for reversing the presbyopia correction, in which the presbyopia correction data is retrieved from the database of the storage device 24. Thus, the original presbyopia correction data is present, with which the laser 12 has generated the multifocal areas.

In a step S14, control data can be ascertained with the aid of the retrieved presbyopia correction data to adapt or cancel the originally generated multifocal areas. Thereto, areas in the cornea 16 can be ascertained based on the retrieved presbyopia correction data, which have not been treated, wherein these areas can then be marked as the tissue to be treated in the control data for compensating for the multifocality. Preferably, these areas are outside of the optical zone 14 such that an original corneal curvature can be restored by removing these areas. Then, it can be provided that the multifocal areas are partially or completely again compensated for, in particular to an original uniform visual acuity. Furthermore, it can additionally be provided, for example if a coma aberration was induced in the original presbyopia correction, that a pupil-to-vertex offset is compensated for based on the presbyopia correction data to undo a coma aberration.

Finally, the thus ascertained control data can be provided for controlling the ophthalmological laser 12 of the treatment apparatus 10 in a step S16. This means that the laser 12 can generate a pattern in the cornea 16 according to the control data, which inverts the original presbyopia correction, and thus a partial or complete original visual acuity is restored.

Overall, the examples show, how a presbyopia reversion to a monofocal cornea can be provided by the invention without using wavefront-guided methods.

1. A method for providing control data for presbyopia reversion for an ophthalmological laser of a treatment apparatus, wherein the method comprises:
determining presbyopia correction data for presbyopia correction of a cornea, wherein an originally uniform visual acuity of the cornea is changed into multifocal areas by application of the presbyopia correction data, and wherein the presbyopia correction data is stored in a database;
if the multifocal areas of the cornea are to be adapted or cancelled at a subsequent point of time, retrieving the stored presbyopia correction data of the cornea from the database;
determining control data for adapting or cancelling the multifocal areas depending on the retrieved presbyopia correction data; and
providing the control data for controlling the ophthalmological laser (12) of the treatment apparatus.

2. The method according to claim 1, wherein the multifocal areas preset from the presbyopia correction data are adapted by at least partially compensating for these multifocal areas.

3. The method according to claim 1, wherein the multifocal areas preset from the presbyopia correction data are adapted by completely compensating for the multifocal areas to the original uniform visual acuity.

4. The method according to claim 1, wherein the presbyopia correction data includes information about an optical zone and a pupil-to-vertex offset.

5. The method according to claim 4, wherein corneal tissue outside of the optical zone is determined in the control data for compensating for multifocal areas.

6. The method according to claim 4, wherein the pupil-to-vertex offset is changed in the control data for compensating for a coma aberration.

7. A method for controlling a treatment apparatus, wherein the method comprises:
the method steps of a method according to claim 1, and
transmitting the provided control data to a respective ophthalmological laser of the treatment apparatus.

8. A control device, which is configured to perform a respective method according to claim 1.

9. A treatment apparatus with at least one ophthalmological laser for the separation of a corneal volume of a human or animal eye by means of optical breakthrough, in particular by means of photodisruption and/or photoablation, and at least one control device according to claim 8.

10. (canceled)

11. A non-transitory computer-readable medium on which a computer program is stored, the computer program including commands that cause at least one control device of a treatment apparatus with at least one ophthalmological laser for the separation of a corneal volume of a human or animal eye by means of optical breakthrough, in particular by means of photodisruption and/or photoablation, to execute a method according to claim 1.
